(19) United States
(12) Patent Application Publication
(10) Pub. No.: US 2007/0167692 A1
(43) Pub. Date: Jul. 19, 2007

(54) Refrigerator with health monitoring system

(76) Inventor: Brian S. Kim, Rancho Palos Verdes, CA (US)

Correspondence Address: PARK LAW FIRM, 3255 WILSHIRE BLVD, SUITE 1110, LOS ANGELES, CA, 90010, US

(21) Appl. No.: 11/333,565

(22) Filed: Jan. 17, 2006

(51) Int. Cl. A61B 5/00; A61B 5/02

(52) U.S. Cl. 600/301; 600/485; 600/549; 600/309

(57) ABSTRACT

A refrigerator with health monitoring system is provided. The refrigerator includes a chamber with a top surface, a bottom surface, a front surface, a rear surface, side surfaces, and a control board on the top surface. The blood pressure measuring device is installed on the control board. The body temperature measuring device is installed on the control board. The blood sugar testing device is installed on the control board. The refrigerator with health monitoring system further includes a main display on the control board of the refrigerator. The main display is adapted to display the test results from the blood pressure measuring device, the body temperature measuring device, and the blood sugar testing device. The main display is turned on by the operation signal from the blood pressure measuring device, the body temperature measuring device, and the blood sugar testing device.

### BACKGROUND OF THE INVENTION

The present invention relates to a refrigerator with a health monitoring system. More particularly, this invention relates to a refrigerator that equipped with a health 10 monitoring system including a blood pressure measuring device, a body temperature measuring device, and a blood sugar testing device.

A refrigerator can be defined as a device that isolates a certain volume of space from the vast environmental space and cools down the temperature in the isolated volume of space lower than the environmental space by means of a well-known heat pump.

The cooled down or temperature-controlled space has been used to store all kinds of food. It is believed that the drastic extension of life expectancy of modern people is mainly thanks to the availability of fresh food and clean water enabled by the introduction of the refrigerator and the running water system.

That is, the space around the refrigerator has become one of the major activity centers of people in a family home.

Health and well-being has been a major concern of the modern people in the industrialized society. Also, the society has become individualized in many aspects. A lot of personal health monitoring systems have been developed in this trends.

The personalized health monitoring system should be stored in a convenient place for everyone to reach and use. Also, it would be much better if some of the features could be shared and exploited fully.

Accordingly, a need for a refrigerator with weighing scale has been present for a long time considering the usage of refrigerators everywhere and the growing interest on health and fitness of general people. This invention is directed to satisfy the long-felt need and to solve some related problems.

### SUMMARY OF THE INVENTION

The present invention contrives to solve the disadvantages of the prior art.

An object of the invention is to provide a refrigerator with health monitoring system.

Another object of the invention is to provide a refrigerator equipped with health monitoring system at a close range.

Still another object of the invention is to provide a refrigerator with health monitoring system that saves space and time.

For the purposes of the invention, a refrigerator with health monitoring system is provided.

The refrigerator includes a chamber with a top surface, a bottom surface, a front surface, a rear surface, side surfaces, and a control board on the top surface.

The blood pressure measuring device is installed on the control board. The body temperature measuring device is installed on the control board. The blood sugar testing device is installed on the control board.

The blood pressure measuring device includes a sphygmomanometer and a blood pressure cuff.

The blood pressure measuring device measures a systolic pressure and a diastolic pressure. The blood pressure measuring device includes a display for displaying the systolic pressure and the diastolic pressure.

The body temperature measuring device includes an electronic clinical thermometer. The electronic clinical thermometer includes an extendable probe and a display.

The body temperature measuring device includes an infrared thermometer. The infrared thermometer includes a laser pointer and a display.

The blood sugar testing device includes a blood sampler and a display.

The refrigerator with health monitoring system further includes a main display on the control board of the refrigerator. The main display is adapted to display the test results from the blood pressure measuring device, the body temperature measuring device, and the blood sugar testing device. The main display is turned on by the operation signal from the blood pressure measuring device, the body temperature measuring device, and the blood sugar testing device.

The refrigerator with health monitoring system further includes a plurality of receptacles adapted for accepting modules for the blood pressure measuring device, the body temperature measuring device, and the blood sugar testing device. The receptacle includes a plurality of electrical contacts between the main display and each of the blood pressure measuring device, the body temperature measuring device, and the blood sugar testing device.

The receptacle includes a locking device and a release switch.

The blood pressure measuring device, the body temperature measuring device, and the blood sugar testing device are modularized and fitted to the corresponding receptacles.

The infrared thermometer with laser pointer is adapted to measure a body temperature of a person in front of the refrigerator in real-time.

The advantages of the present invention are: (1) the refrigerator with health monitoring system is convenient to use a couple of types of health monitoring system in one place; (2) the refrigerator with health monitoring system helps people who are interested in health; and (3) the refrigerator with health monitoring system provides an easy way to read the test results from the health monitoring system.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view of a refrigerator with a weighing scale extracted from under the refrigerator according to the present invention; and

FIG. 2 is a front plan view of a control board of the refrigerator according to the present invention.

### DETAILED DESCRIPTION OF THE INVENTION

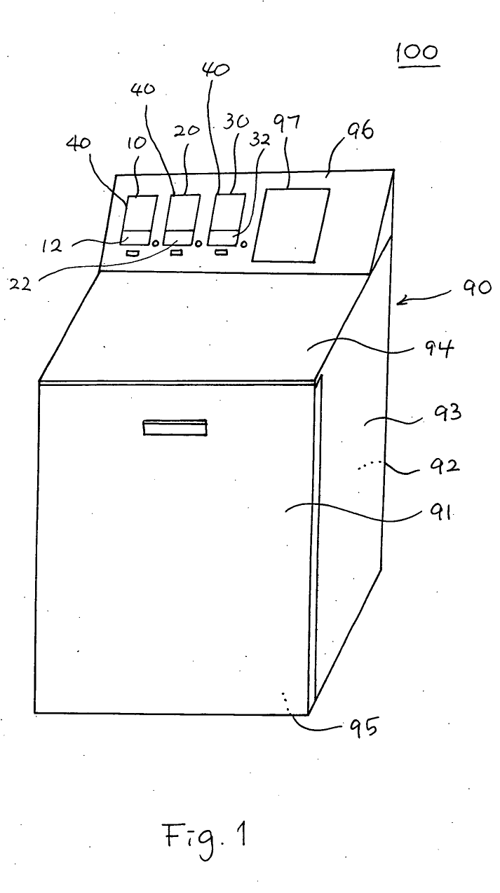

FIG. 1 shows a refrigerator with health monitoring system 100.

The refrigerator with health monitoring system 100 includes a refrigerator 90, a blood pressure measuring device 10, a body temperature measuring device 20, and a blood sugar testing device 30.

The refrigerator 90 includes a chamber with a top surface 94, a bottom surface 95, a front surface 91, a rear surface 92, side surfaces 93, and a control board 96 on the top surface 94.

The blood pressure measuring device 10 is installed on the control board 96. The body temperature measuring device 20 is installed on the control board 96. The blood sugar testing device 30 is installed on the control board 96.

The blood pressure measuring device 10 includes a sphygmomanometer and a blood pressure cuff.

The blood pressure measuring device 10 measures a systolic pressure and a diastolic pressure. The blood pressure measuring device 10 includes a display 12 for displaying the systolic pressure and the diastolic pressure.

The body temperature measuring device 20 includes an electronic clinical thermometer. The electronic clinical thermometer includes an extendable probe (not shown) and a display 22.

The body temperature measuring device 20 includes an infrared thermometer. The infrared thermometer includes a laser pointer (not shown) and a display 22.

The blood sugar testing device 30 includes a blood sampler (not shown) and a display 32.

The refrigerator with health monitoring system 100 further includes a main display 97 on the control board 96 of the refrigerator 90. The main display 97 is adapted to display the test results from the blood pressure measuring device 10, the body temperature measuring device 20, and the blood sugar testing device 30. The main display 97 is turned on by the operation signal from the blood pressure measuring device 10, the body temperature measuring device 20, and the blood sugar testing device 30.

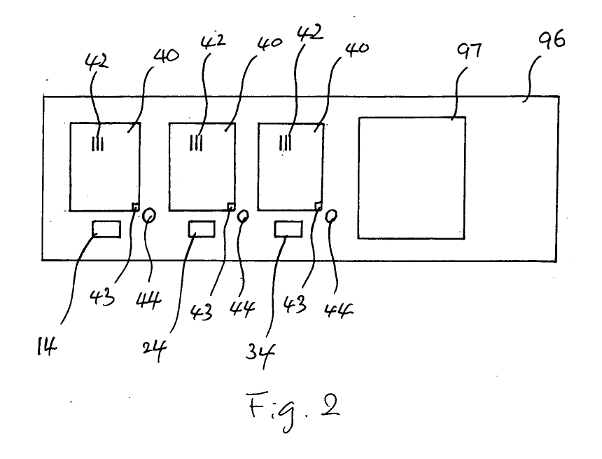

As shown in FIG. 2, the refrigerator with health monitoring system 100 further includes a plurality of receptacles 40 adapted for accepting modules for the blood pressure measuring device 10, the body temperature measuring device 20, and the blood sugar testing device 30. The receptacle 40 includes a plurality of electrical contacts 42 inside between the main display 97 and each of the blood pressure measuring device 10, the body temperature measuring device 20, and the blood sugar testing device 30.

The receptacle 40 includes a locking device 43 and a release switch 44.

The blood pressure measuring device 10, the body temperature measuring device 20, and the blood sugar testing device 30 are modularized and fitted to the corresponding receptacles 40.

Each of the blood pressure measuring device 10, the body temperature measuring device 20, and the blood sugar testing device 30 may include corresponding operation switch 14, 24, 34 as shown in FIG. 2.

The infrared thermometer with laser pointer is adapted to measure a body temperature of a person in front of the refrigerator 90 in real-time.

The sphygmomanometer and blood pressure cuff for the blood pressure measuring device 10, the electronic clinical thermometer or the infrared thermometer for the body temperature measuring device 20, and the blood sugar testing device 30 are available in the market.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A refrigerator with health monitoring system comprising:
a) a refrigerator comprising a chamber with a top surface, a bottom surface, a front surface, a rear surface, side surfaces, and a control board on the top surface; and
b) a blood pressure measuring device installed on the control board;
c) a body temperature measuring device installed on the control board; and
d) a blood sugar testing device installed on the control board.

2. The refrigerator with health monitoring system of claim 1, wherein the blood pressure measuring device comprises a sphygmomanometer and a blood pressure cuff.

3. The refrigerator with health monitoring system of claim 1, wherein the blood pressure measuring device measures a systolic pressure and a diastolic pressure.

4. The refrigerator with health monitoring system of claim 3, wherein the blood pressure measuring device comprises a display for displaying the systolic pressure and the diastolic pressure.

5. The refrigerator with health monitoring system of claim 1, wherein the body temperature measuring device comprises an electronic clinical thermometer.

6. The refrigerator with health monitoring system of claim 5, wherein the electronic clinical thermometer comprises an extendable probe and a display.

7. The refrigerator with health monitoring system of claim 1, wherein the body temperature measuring device comprises an infrared thermometer.

8. The refrigerator with health monitoring system of claim 7, wherein the infrared thermometer comprises a laser pointer and a display.

9. The refrigerator with health monitoring system of claim 1, wherein the blood sugar testing device comprises a blood sampler and a display.

10. The refrigerator with health monitoring system of claim 1, further comprising a main display on the control board of the refrigerator.

11. The refrigerator with health monitoring system of claim 10, wherein the main display is adapted to display the test results from the blood pressure measuring device, the body temperature measuring device, and the blood sugar testing device.

12. The refrigerator with health monitoring system of claim 11, wherein the main display is turned on by the operation signal from the blood pressure measuring device, the body temperature measuring device, and the blood sugar testing device.

13. The refrigerator with health monitoring system of claim 1, further comprising a plurality of receptacles adapted for accepting modules for the blood pressure measuring device, the body temperature measuring device, and the blood sugar testing device.

14. The refrigerator with health monitoring system of claim 13, wherein the receptacle comprises a plurality of electrical contacts between the main display and each of the blood pressure measuring device, the body temperature measuring device, and the blood sugar testing device.

15. The refrigerator with health monitoring system of claim 13, wherein the receptacle comprises a locking device and a release switch.

16. The refrigerator with health monitoring system of claim 13, wherein the blood pressure measuring device, the body temperature measuring device, and the blood sugar testing device are modularized and fitted to the corresponding receptacles.

17. The refrigerator with health monitoring system of claim 16, wherein each of the blood pressure measuring device, the body temperature measuring device, and the blood sugar testing device further comprises a corresponding operation switch provided on the control board of the refrigerator.

18. The refrigerator with health monitoring system of claim 1, wherein the body temperature measuring device comprises an infrared thermometer with laser pointer, wherein the infrared thermometer with laser pointer is adapted to measure a body temperature of a person in front of the refrigerator in real-time.
